(19) United States
(12) Patent Application Publication
(10) Pub. No.: US 2016/0361235 A1
(43) Pub. Date: Dec. 15, 2016

(54) ADAPTER ASSEMBLY FOR ENTERAL FEEDING

(71) Applicant: Covidien LP, Mansfield, MA (US)

(72) Inventor: David Rork Swisher, St. Charles, MO (US)

(21) Appl. No.: 15/174,357

(22) Filed: Jun. 6, 2016

Related U.S. Application Data
(60) Provisional application No. 62/174,493, filed on Jun. 11, 2015.

(51) Int. Cl. A61J 15/00; A61M 39/10; A61M 39/12

(52) U.S. Cl. CPC A61J 15/0026; A61M 39/12; A61M 39/1011; A61M 2039/1077; A61M 2202/0482; A61M 2039/1033

(57) ABSTRACT

An enteral feeding adapter comprising a male fitting configured to engage a female fitting for connecting the adapter to an enteral feeding element. The male fitting includes an annular portion having a threaded inner surface and a tubular portion disposed at least partially within the annular portion. The tubular portion includes a side wall, an outer diameter, and a central passage extending through the tubular portion. The side wall of the tubular portion extending past an open end of the annular portion for use in filling an interior space of a female fitting when received in the female fitting.

### CROSS-REFERENCE TO RELATED APPLICATION

This application claims the benefit of priority under 35 U.S.C. §119 to U.S. Patent Application No. 62/174,493, titled ADAPTER ASSEMBLY FOR ENTERAL FEEDING, which was filed on Jun. 11, 2015, and which is incorporated herein by reference in its entirety for all purposes.

### FIELD

The present disclosure generally relates to an adapter assembly for enteral feeding.

### BACKGROUND

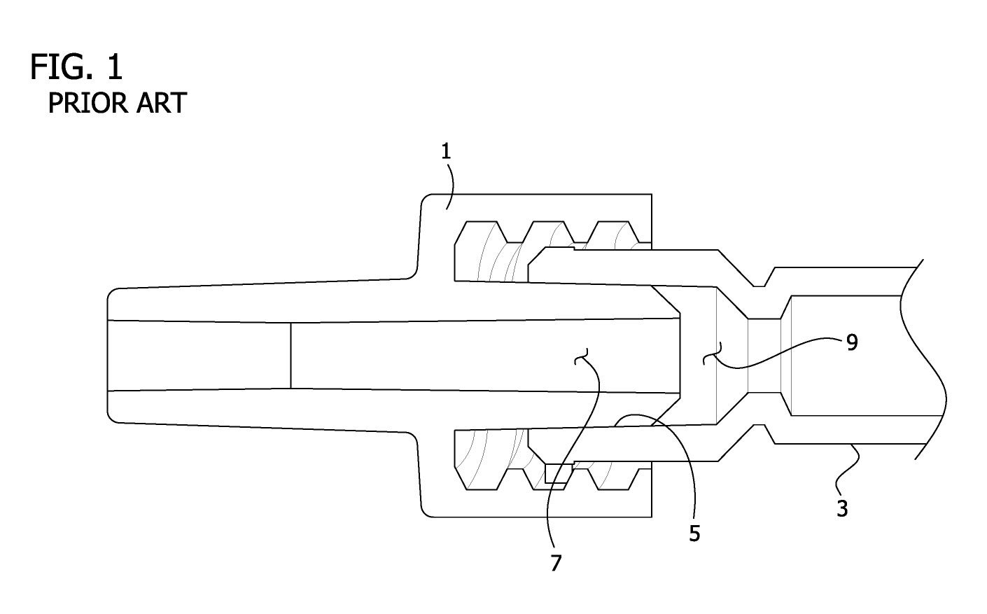

FIG. 1 shows a conventional enteral feeding connector assembly of the prior art including a male enteral feeding connector 1 and a female enteral feeding connector 3 configured to be connected to one another for use in connecting enteral fluid lines in healthcare applications. The female enteral feeding connector 3 receives a portion of the male enteral feeding connector 1 within an interior space 5. The enteral feeding connectors 1, 3 deliver fluid in the fluid lines through a fluid passage 7 extending through the connectors. Fluid is typically delivered from the female connector 3 to the male connector 1. A dead space 9 is located adjacent an inlet of the male enteral feeding connector 1. The dead space 9 defines an area of the interior space 5 in the female enteral feeding connector 3 that is not occupied by the portion of the male enteral feeding connector 1 received therein. A problem with having this dead space is that fluid intended for delivery can pool within the dead space causing an under dose of fluid to be delivered to the patient. Conversely, the pooled fluid may not be accounted for in the fluid measurement resulting in an overdose of fluid to the patient. Although errors may be small, they can be important for certain patients, such as neo-natal infants.

### SUMMARY

In one aspect, an enteral feeding adapter generally comprises a male fitting configured to engage a female fitting for connecting the adapter to an enteral feeding element. The male fitting includes an annular portion having a threaded inner surface and a tubular portion disposed at least partially within the annular portion. The tubular portion includes a side wall, an outer diameter, and a central passage extending through the tubular portion. The side wall of the tubular portion extends past an open end of the annular portion by a distance. A ratio of the outer diameter of the tubular portion to the distance the side wall of the tubular portion extends past the open end of the annular portion is between about 1 to 1 and about 2 to 1.

In another aspect, an enteral feeding adapter generally comprises a male fitting configured to engage a female fitting for connecting the adapter to an enteral feeding element. The male fitting includes an annular portion having a threaded inner surface and a tubular portion disposed at least partially within the annular portion. The tubular portion including an annular side wall, a beveled end wall, and a central passage extending through the tubular portion. The annular side wall of the tubular portion extending past an open end of the annular portion.

In yet another aspect, an enteral feeding adapter assembly generally comprises a male connector configured for connection to medical tubing. The male connector comprises a male fitting including an annular portion having a threaded inner surface, a tubular portion disposed at least partially within the annular portion, and a central passage extending through the tubular portion. A female fitting is configured for connection to the male fitting and to an enteral feeding element. The female fitting comprises an annular portion defining an interior space for receiving the tubular portion of the male fitting when the male connector is connected to the female fitting. The tubular portion is configured to substantially fill the interior space when the male connector and female fitting are connected.

### BRIEF DESCRIPTION

FIG. 1 is a cross section of a prior art enteral feeding connection assembly;

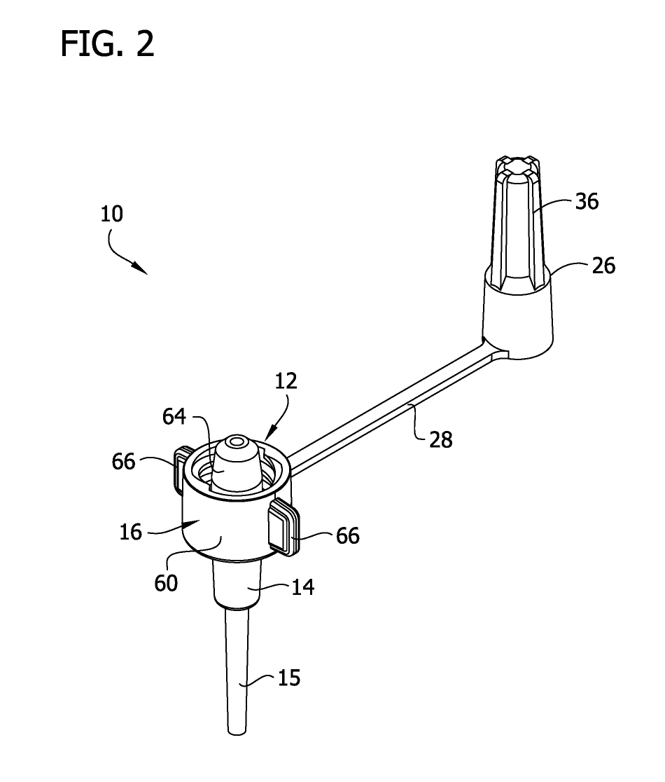

FIG. 2 is a perspective of an adapter assembly;

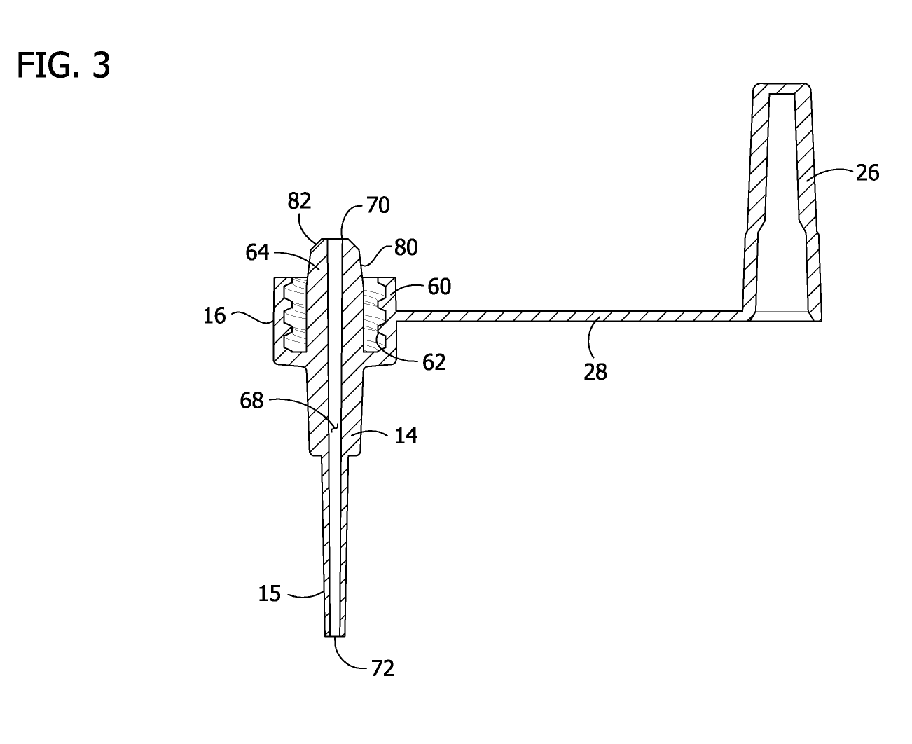

FIG. 3 is a vertical section of the adapter assembly;

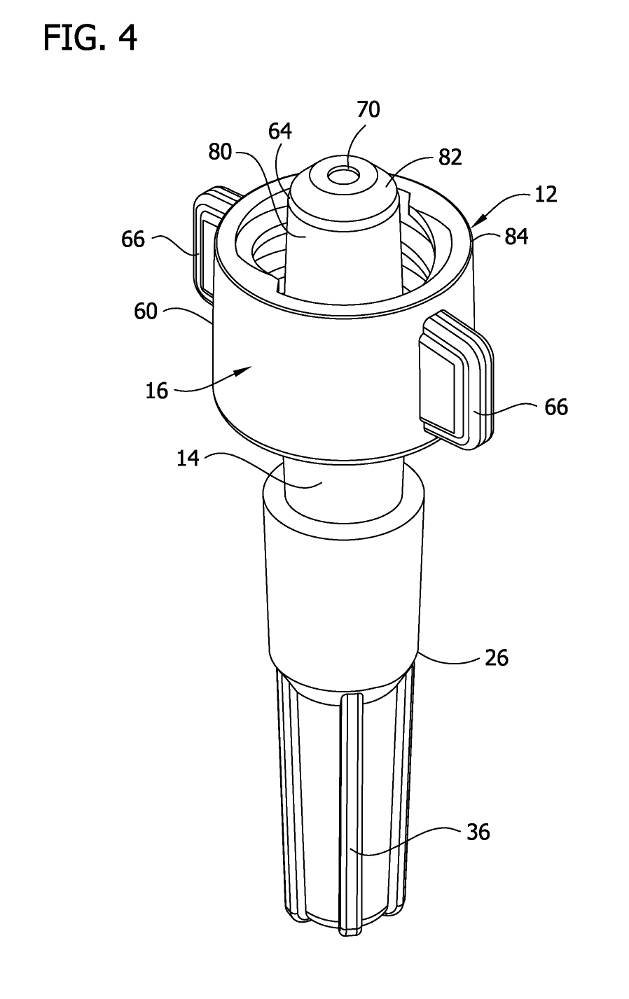

FIG. 4 is a perspective of the adapter assembly with a cap secured around a flexible member of the assembly;

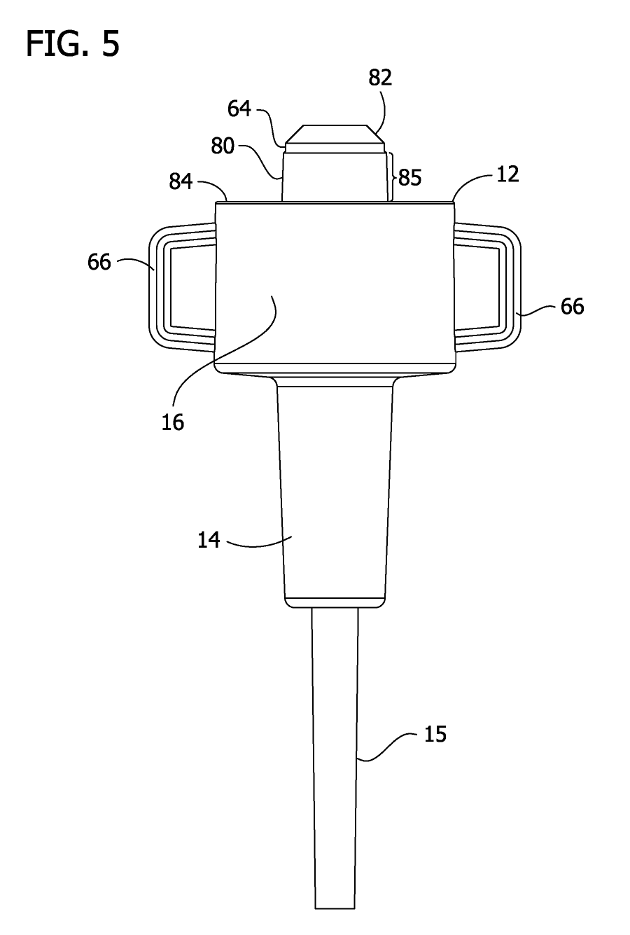

FIG. 5 is a front view of the adapter assembly with the cap removed;

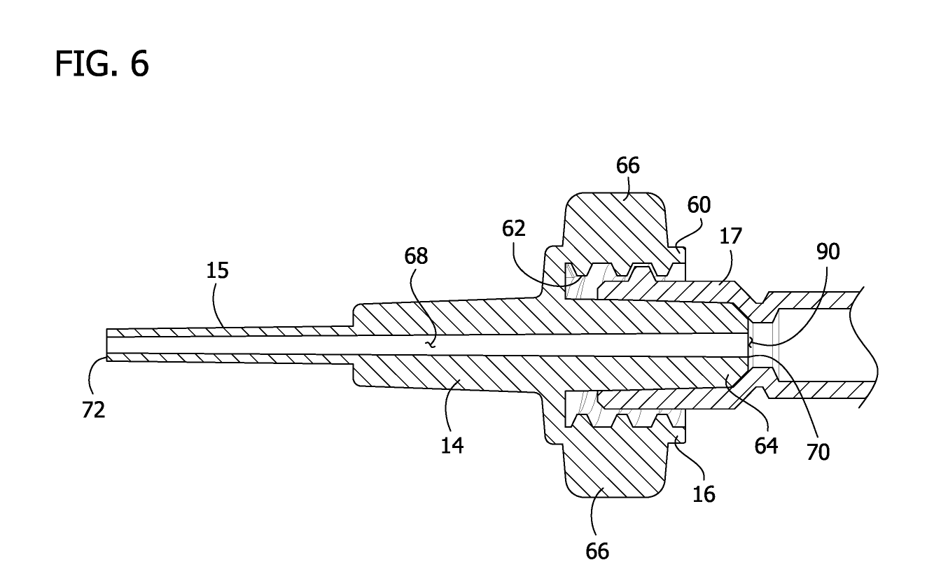

FIG. 6 is a section of the assembly connected to a mating connector;

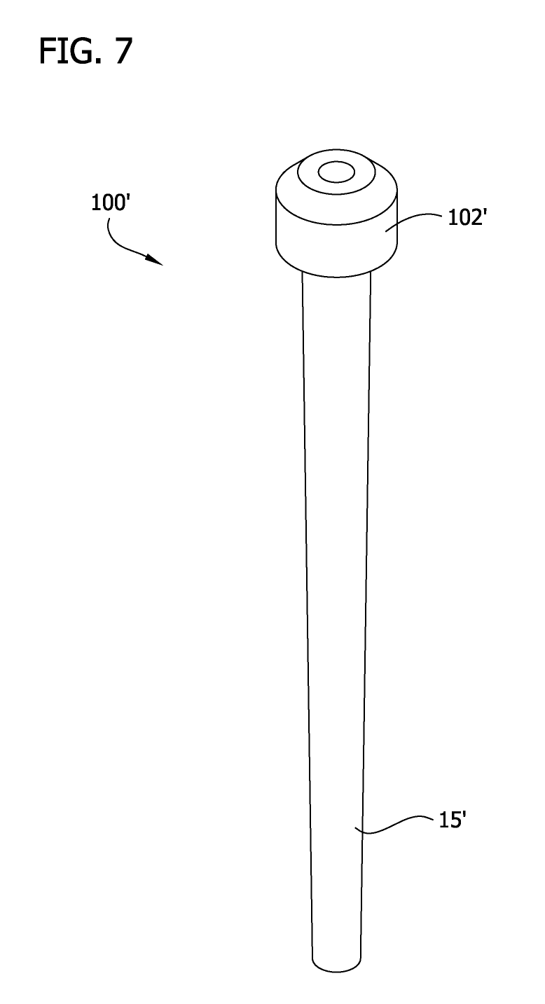

FIG. 7 is a perspective of an insert; and

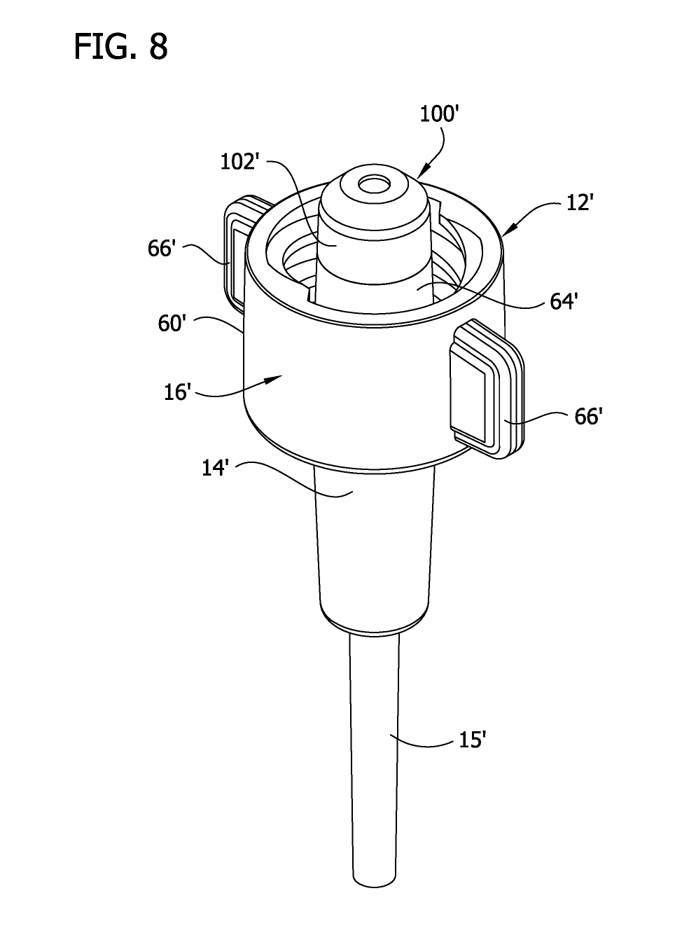

FIG. 8 is a perspective of an adapter assembly of a second embodiment including the insert.

Corresponding reference characters indicate corresponding parts throughout the drawings.

### DETAILED DESCRIPTION OF THE DRAWINGS

Referring to FIG. 2, an enteral feeding adapter assembly is generally indicated at 10. The adapter assembly is configured to fluidly connect an enteral feeding element (e.g., syringe, feeding bag, pump, etc.) to a fluid conduit or reservoir (e.g., medical tubing, bottle, etc.) for delivering fluid to a subject through the fluid conduit or retrieving fluid from the fluid reservoir. The adapter assembly 10 includes an enteral feeding element connection portion 12 for connecting to an enteral feeding element (not shown) and a fluid conduit/reservoir connection portion 14 for connecting to a fluid conduit/reservoir (not shown). A flexible member 15 extends from an end of the fluid conduit/reservoir connection portion 14. As will be explained in greater detail below, the enteral feeding element connection portion 12 may include a male fitting 16 for connecting to a complementary female fitting 17 (FIG. 6) associated with the enteral feeding element. In one embodiment, the female fitting 17 may be part of a syringe. The male fitting 16 is configured to substantially eliminate a dead space between the male fitting and the female fitting 17 when the fittings are connected. As a result, fluid cannot pool in the female fitting at an inlet of the male fitting 16 as can be the case for the connector assembly of the prior art in FIG. 1. As will be explained in greater detail below, this facilitates a more accurate delivery of enteral fluid to the subject. While the female fitting 17 is illustrated as separate from the enteral feeding adapter assembly 10, it is envisioned that the female fitting could be part of the adapter assembly.

In the illustrated embodiment, the connection portion 14 and flexible member 15 are formed integrally with the male fitting 16. In the preferred embodiment, the connection portion 14, flexible member 15, and male fitting 16 may be molded as one plastic piece from a suitable material such as a thermoplastic polymer. However, the connection portion 14 could be formed separately from the male fitting 16 and attached to the male fitting by a suitable means. Also, the flexible member 15 could be formed separately from the connection portion 14 and attached to the connection portion by a suitable means. This particular embodiment is shown in FIGS. 7 and 8 and will be discussed in greater detail below. The flexible member 15 can be molded such that it is more flexible than the connection portion 14. The connection portion 14, flexible member 15, and male fitting 16 could also be formed from a material other than plastic. A cap 26 may be secured to the male fitting 16 by a tether 28. The cap 26 may be configured to slide over the flexible member 15 when the adapter assembly 10 is not in use to receive the flexible member and at least a portion of the connection portion 14 therein (FIG. 4). The cap 26 may include a grip portion 36 for manipulating the cap. The flexible member 15 can be positioned at the bottom of a medicine vial (not shown) for purposes of filling a syringe connected to the feeding adapter 10. The flexible member is purposefully small in diameter so that another tube or connector cannot fit inside it, which may allow for a misconnection. Moreover if the flexible portion 15 were rigid, it might be used like a blunt needle. Still further, the flexible tube also allows for oral administration.

Referring to FIGS. 2-5, the male fitting 16 includes an annular portion 60 having a threaded inner surface 62 and a tubular portion 64 disposed at least partially within the annular portion. The threaded inner surface 62 and an outer surface of the tubular portion 64 are configured to engage female fitting 17 for connecting the adapter assembly 10 to an enteral feeding element (FIG. 6). Wings 66 on the male fitting 16 facilitate rotation of the male fitting when it is engaged with the female fitting 17. In the illustrated embodiment, the wings 66 are disposed on opposite sides of the male fitting 16. A central passage 68 extends through the male fitting 16, the connection portion 14, and the flexible member 15. The central passage 68 extends between an inlet 70 of the male fitting 16 and an outlet 72 of the flexible member 15. In one embodiment, the male fitting 16, connection portion 14, and flexible member 15 are coaxial. The male fitting 16, connection portion 14, and flexible member 15 may have a combined length in a range from about 25 mm to about 100 mm, and the central passage 68 may have a diameter of about 2.8 mm. The outer diameter of the tubular portion 64 may be less than about 5.46 mm, e.g., about 5.41 mm, and the outer diameter of the annular portion 60 (not including wings 66) may be about 12.2 mm. The connection portion 14 may taper from a widest portion (nearest male fitting 16) to a narrowest portion (nearest flexible member 15). In one embodiment, the widest outer diameter of the connection portion 14 may be about 6.2 mm and the narrowest portion may have an outer diameter of about 5.1 mm. The flexible member 15 may have an outer diameter that is smaller than the outer diameter of the narrowest portion of the connection portion 14. In one embodiment, the flexible member 15 has an outer diameter of about 2.85 mm. It will be understood that the dimensions are exemplary only and that the connection portion may have other dimensions within the scope of the present invention.

The tubular portion 64 includes an annular side wall 80 and a beveled end wall 82. The tubular portion 64 extends past a rim 84 of the annular portion 60 such that an end margin 85 of the side wall extends past the rim of the annular portion. In the illustrated embodiment a length of the end margin 85 (FIG. 5) is less than about 3.5 mm, e.g., about 2 mm. In one embodiment, a ratio of the outer diameter of the tubular portion 64 to the length of the end margin 85 is between about 1 to 1 and about 2 to 1. In a preferred embodiment, the ratio is about 1.5 to 1.

Referring to FIG. 6, in use, the adapter assembly 10 places a syringe or other device having a female fitting 17 in fluid communication with a feeding tube or other fluid conduit. Alternatively, the adapter assembly 10 can place the syringe or other device having a female fitting 17 in fluid commination with a fluid reservoir (e.g., nutrient bag) for withdrawing fluid from the fluid reservoir. In the case of fluid delivery to a fluid conduit, fluid from the fluid source flows past the inlet 70 of the male fitting 16, through the central passage 68 and out the outlet 72 of the flexible member 15 and into the fluid conduit.

When the adapter assembly 10 is attached to the female fitting 17, the tubular portion 64 of the male fitting 16 is received in an interior space 90 in the female fitting (FIG. 6). The construction of the tubular portion 64 of the male fitting 16 is such that the tubular portion occupies substantially all of the interior space 90 in the female fitting 17. For example, the portion 64 may have a volume that is in a range of from about 70% to 95% of the volume of the dead space 9. Thus, the adapter assembly 10 substantially eliminates the dead space that is present in connection assemblies of the prior art (FIG. 1). As a result, fluid is not permitted to pool in the interior space 90 of the female fitting 17 at the inlet 70 of the male fitting 16. By filling up substantially all of the female fitting 17 of a syringe having the interior space 90, the quantity of fluid delivered from or taken into the syringe can be very precise. This is because the dead space 9 (FIG. 1) that would exist between the end of the male connector and the bottom of the female connector is substantially eliminated.

While the tubular portion 64 has been shown as having an entirety formed integrally with the connection portion 14 and annular portion 60, an insert 100′ (FIG. 7) may be provided for modifying a conventional male connector as shown in FIG. 1 to have a configuration similar to that of the adapter assembly 10 in the previous embodiment. The insert 100′ may include a tubular extension element 102′ and a flexible member 15′ extending from the tubular extension element. Referring to FIG. 8, the flexible member 15′ may be inserted into an inlet of tubular portion 64′ such that the flexible member 15′ extends through the tubular portion and a connection portion 14′. The flexible member 15′ is sized so that a length of the flexible member is sufficient to extend from the connection portion 14′ to substantially the same extent that flexible member 15 extends from connection portion 14 in the previous embodiment. The tubular extension element 102′ extends the length of the tubular portion 64′ so that the configuration of the tubular portion 64′ and extension element 102′ is substantially the same as the tubular portion 64 of the previous embodiment. In the illustrated embodiment, the extension element 102′ extends seamlessly from the tubular portion 64′. However, the extension element 102′ could extend from the tubular portion 64′ at an angle such that a break is formed between the tubular portion 64′ and the extension element 102′. The insert 100′ may be formed of a flexible material so that the insert is more flexible than at least the tubular portion 64′ and connection portion 14′. As a result, the more rigid tubular portion 84′ performs a sealing function while the extension element 102′ fills a dead space that would otherwise exist in the interior space of the syringe.

Having described embodiments of the invention in detail, it will be apparent that modifications and variations are possible without departing from the scope of the invention defined in the appended claims.

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

1. An enteral feeding adapter comprising a male fitting configured to engage a female fitting for connecting the adapter to an enteral feeding element, the male fitting including an annular portion having a threaded inner surface and a tubular portion disposed at least partially within the annular portion, the tubular portion including a side wall, an outer diameter, and a central passage extending through the tubular portion, the side wall of the tubular portion extending past an open end of the annular portion by a distance, a ratio of the outer diameter of the tubular portion to the distance the side wall of the tubular portion extends past the open end of the annular portion is between about 1 to 1 and about 2 to 1.

2. The adapter set forth in claim 1 wherein the ratio is about 1.5 to 1.

3. The adapter set forth in claim 1 further comprising a tube connection portion extending from a closed end of the annular portion and a flexible member extending from the tube connection portion.

4. The adapter set forth in claim 3 further comprising a passage extending through the tubular portion and the flexible portion.

5. The adapter set forth in claim 3 wherein the annular portion, tubular portion, tube connection portion, and flexible member are coaxial.

6. The adapter set forth in claim 3 further comprising a cap tethered to the annular portion, the cap being configured to receive the flexible member and at least a portion of the tube connection portion.

7. The adapter set forth in claim 3 wherein the annular portion, tubular portion, tube connection portion, and flexible member are formed as one piece of material.

8. The adapter set forth in claim 3 wherein the flexible member has a blunt free end.

9. The adapter set forth in claim 3 further comprising an insert received in the tubular portion and tube connection portion, the insert defining the flexible member and a portion of the tubular portion.

10. The adapter set forth in claim 9 wherein the insert is more flexible than the tube connection portion.

11. The adapter set forth in claim 9 wherein the insert defines the side wall of the tubular portion that extends past the open end of the annular portion.

12. An enteral feeding adapter comprising a male fitting configured to engage a female fitting for connecting the adapter to an enteral feeding element, the male fitting including an annular portion having a threaded inner surface and a tubular portion disposed at least partially within the annular portion, the tubular portion including an annular side wall, a beveled end wall, and a central passage extending through the tubular portion, the annular side wall of the tubular portion extending past an open end of the annular portion.

13. The adapter set forth in claim 12 wherein the annular side wall extends past the open end of the annular portion by about 2 mm.

14. The adapter set forth in claim 12 further comprising a tube connection portion extending from a closed end of the annular portion and a flexible member extending from the tube connection portion.

15. The adapter set forth in claim 14 wherein the annular portion, tubular portion, tube connection portion, and flexible member are coaxial.

16. The adapter set forth in claim 14 further comprising a cap tethered to the annular portion, the cap being configured to receive the flexible member and at least a portion of the tube connection portion.

17. The adapter set forth in claim 14 wherein the annular portion, tubular portion, tube connection portion, and flexible member are formed as one piece of material.

18. The adapter set forth in claim 14 further wherein the central passage extends through the flexible member.

19. An enteral feeding adapter assembly comprising:
a male connector configured for connection to medical tubing, the male connector comprising a male fitting including an annular portion having a threaded inner surface, a tubular portion disposed at least partially within the annular portion, and a central passage extending through the tubular portion; and
a female fitting configured for connection to the male fitting and to an enteral feeding element, the female fitting comprising an annular portion defining an interior space for receiving the tubular portion of the male fitting when the male connector is connected to the female fitting, the tubular portion being configured to substantially fill the interior space when the male connector and female fitting are connected.

20. The adapter assembly set forth in claim 19 wherein the tubular portion fills at least about 70% of the interior space of the female fitting.
